(19) United States
(12) Patent Application Publication
(10) Pub. No.: US 2006/0025654 A1
(43) Pub. Date: Feb. 2, 2006

(54) Endoscopic system for treating inside of body cavity

(75) Inventors: Keita Suzuki, Kokubunji-shi (JP); Tsuyoshi Tsukagoshi, Fuchu-shi (JP); Ryuta Sekine, Koganei-shi (JP); Anthony Nicholas Kalloo, Glenn Dale, MD (US); Sergey Veniaminovich Kantsevoy, Silver Spring, MD (US)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, TOKYO (JP)

(21) Appl. No.: 11/236,138

(22) Filed: Sep. 27, 2005

Related U.S. Application Data
(62) Division of application No. 10/390,364, filed on Mar. 17, 2003.
(60) Provisional application No. 60/365,266, filed on Mar. 18, 2002.

(51) Int. Cl. A61B 1/00; A61M 29/00

(52) U.S. Cl. 600/114; 600/115; 600/104; 606/192

(57) ABSTRACT

There is provided an endoscopic inserting system. The system includes an endoscope which is inserted into a lumen inside a body through a natural opening of a human body, an opening member which forms an opening for inserting the endoscope into a thoracic cavity or an abdominal cavity from the lumen inside the body at a wall portion of the lumen, and a retracting member which, when forming the opening, retracts the wall portion of the lumen.

### CROSS-REFERENCE TO RELATED APPLICATIONS

This application claims the benefit of U.S. Provisional Application No. 60/365,266, filed Mar. 18, 2002.

### BACKGROUND OF THE INVENTION

The present invention relates to an endoscopic system for observing and/or treating the inside of the abdominal cavity and/or thoracic cavity by inserting the system into the human body, and a treatment device applicable thereto.

In general, there are a variety of systems for inserting an endoscope through a natural opening in the human body, dissecting a lumen, and then, treating the inside of the abdominal cavity.

For example, in U.S. Pat. No. 5,297,536, a treatment system as shown in FIG. 34 is disclosed. This system is composed of a dissecting device for perforating a lumen wall; an endoscope insert member for inserting an endoscope, a tube, an endoscope, and a pneumoperitoneum device for deflating the abdominal cavity; and a closing device.

When surgery of the inside of the abdominal cavity is carried out using this system, the endoscope insert member and tube are first inserted through a natural opening in the human body and the tube absorbed to a required organ wall by vacuum pressure, thus being fixed thereon. Next, the pneumoperitoneum needle is inserted and the lumen is subjected to pneumoperitoneum. Then, the dissecting device is inserted and the organ wall is perforated. After surgery of the inside of abdominal cavity is complete, the perforation in the organ wall is closed by an O-ring, and the endoscope and tube are withdrawn from the body. In this system, it is difficult to dissect only the lumen wall to be separated from the organ adjacent to the lumen wall when perforating the lumen wall.

In addition, an endoscopic treatment device as shown in FIG. 35 is disclosed in Jpn. Pat. Appln. KOKAI Publication No. 2001-9037. This treatment device is formed as a balloon catheter that consists of an elongated sheath inserted into an endoscope; a balloon placed at the outer periphery of the sheath distal end portion; and a port mounted in front of the sheath.

A stenosis site of a living tissue is dilated by using this balloon catheter as follows.

First, the balloon catheter is inserted into the body from the forceps opening of the endoscope through the channel, and then is inserted into the stenosis site under observation of the endoscope. Next, an inflation device is connected to the port, fluid is supplied to the balloon, and the balloon is dilated. After the balloon has been dilated over a sufficient time to a sufficient dilation diameter, the balloon is contracted and withdrawn from the body, terminating treatment. However it is difficult to position this balloon catheter at a required location under the endoscope.

In addition, an endoscopic treatment device as shown in FIG. 36 is disclosed in Jpn. Pat. Appln. KOKAI Publication No. 2000-51361. This treatment device is formed as a balloon catheter that consists of: an elongated sheath inserted into the endoscope; a balloon placed at the outer periphery of the sheath distal end portion; a port attached to the sheath proximal portion; and two markings provided on the sheath at the portion at which the balloon is placed.

A stenosis site of a living tissue is dilated by using this balloon catheter as follows.

First, the balloon catheter is inserted into the inside of the body from the forceps opening of the endoscope through the channel, and then, is inserted into the stenosis site under observation by the endoscope in accordance with the markings. Next, an inflation device is connected to the port, fluid is supplied to the balloon, and the balloon is dilated. After the balloon has been dilated for a sufficient time to a sufficient dilation diameter, the balloon is contacted and withdrawn from the body, terminating treatment. However when treatment is carried out by using this balloon catheter, the balloon is loaded on the markings, thus making it difficult to clearly see the markings.

Further, a high-frequency catheter as shown in FIG. 37 is described in British Pat. Appln. Publication No. 2 145 932 A. This high-frequency catheter is composed of an elongated shaft to be inserted into a blood vessel; a high-frequency surgical knife provided to freely extend and retract at the distal end of the shaft; a balloon; a valve for the balloon; and a high-frequency knife manipulating portion.

Coagulation work in a blood vessel is carried out by using this high-frequency catheter as follows. The high-frequency catheter is inserted into a vein, and the vein is heated with a high-frequency, and then dilated by the balloon. After the vein has sufficiently dilated, the balloon is contracted and is withdrawn from the body. This high-frequency catheter is believed to have the possibility of the balloon slipping during dilation.

Further, a high-frequency catheter as shown in FIG. 38 is described in U.S. Pat. No. 6,093,187. This high-frequency catheter is composed of an elongated shaft to be inserted into a brain chamber; a high-frequency surgical knife provided at the distal end of the shaft; a balloon dilator placed at the shaft; a valve for the balloon; and a plug for the high-frequency surgical knife.

When perforating/dilation work on the inside of the brain chamber is carried out using this high-frequency catheter, a membrane is perforated with the high-frequency surgical knife, and the catheter is inserted. Next, the perforation is dilated by the balloon dilator. Because an electrode always protrudes, proper insertion properties relevant to a physiological wall cannot be obtained.

### BRIEF SUMMARY OF THE INVENTION

The present invention has been made in view of the above-described circumstances. It is an object of the present invention to provide an endoscopic system for perforating a lumen wall reliably and safely and a treatment device applicable thereto.

Further, it is an object of the present invention to facilitate positioning relevant to a lumen wall of a balloon dilator contracted in diameter at its center portion.

In order to achieve the foregoing object, according to a first aspect of the present invention, there is provided an endoscopic inserting system. The system includes an endoscope which is inserted into a lumen inside a body through a natural opening of a human body, an opening member which forms an opening for inserting the endoscope into a thoracic cavity or an abdominal cavity from the lumen inside the body at a wall portion of the lumen, and a retracting member which, when forming the opening, retracts the wall portion of the lumen.

Preferably, the retracting member comprises: a sucker which provides a negative pressure; and a tubular member to maintain a negative pressure at a region in which the opening member acts.

According to another aspect of the present invention, there is provided an endoscopic inserting system comprising: an endoscope which is capable of being inserted into a lumen inside a body through a natural opening of a human body; an over-tube through which the endoscope is capable of being inserted; and an introducer into which the endoscope inserted through the over-tube is capable of being inserted.

According to still another aspect of the present invention, there is provided a balloon dilator comprising: a distal end portion; a center portion; a proximal end portion; a distal maximum external diameter portion disposed at a distal side more than the center portion, the distal maximum external diameter portion having a diameter which is greater than the center portion; a proximal maximum external diameter portion disposed at a proximal side more than the center portion, the proximal maximum external diameter portion having a diameter which is greater than the center portion; a surface portion; and a marker provided at the surface portion.

Advantages of the present invention will be set forth in the description which follows, and in part will be obvious from the description, or may be learned by practice of the present invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate presently preferred embodiments of the present invention, and together with the general description given above and the detailed description of the preferred embodiments given below, serve to explain the principles of the present invention.

FIG. 1 is a view illustrating a state in which surgical operation is carried out by using an endoscopic system for treating the inside of a body cavity according to a first embodiment of the present invention;

FIG. 2 is a view illustrating an endoscope shown in FIG. 1;

FIG. 2A is an enlarged view showing a distal end portion of the endoscope of FIG. 1;

FIG. 3 is a view showing an entire structure of an opening treatment device used for the system of FIG. 1;

FIG. 4 is an enlarged view showing a distal end portion of the opening treatment device of FIG. 3;

FIG. 5 to FIG. 7 are views each showing a variety of modified examples of a balloon shown in FIG. 4;

FIG. 8 is a sectional view showing an internal structure of a distal end portion of the opening treatment device of FIG. 3 while a high-frequency surgical knife is extended;

FIG. 9 is a sectional view similar to FIG. 8 while the high-frequency surgical knife is retracted;

FIG. 10 is a sectional view showing an opening treatment device while a guide wire is inserted;

FIG. 11 is a view illustrating a state in which the endoscope of FIG. 1 having a transparent cap mounted thereon approaches a lumen wall;

FIG. 12 is a view illustrating a state in which the lumen wall is suctioned;

FIG. 13 is a view illustrating a state in which a high-frequency surgical knife is pierced into a lumen wall while the lumen wall is suctioned;

FIG. 14 is a view illustrating a state in which an opening treatment device is inserted into an abdominal cavity through an opening in the lumen wall formed in FIG. 13;

FIG. 15 is a view illustrating a state in which an opening of the lumen wall formed in FIG. 13 is dilated by means of a balloon dilator;

FIG. 16 is a view illustrating a state in which the endoscope approaches the inside of abdominal cavity through the opening formed in the lumen wall;

FIG. 17 is an external view showing an endoscopic system for treating the inside of a body cavity according to a second embodiment when the endoscope and over-tube are combined with each other;

FIG. 18 is an external view showing an over-tube simplex shown in FIG. 17;

FIG. 19 is a view illustrating a surgical operation using a system of FIG. 18;

FIG. 20 is a sectional view showing a distal end portion of an opening treatment device according to a third embodiment;

FIG. 21 is an external view showing an entire system according to a fourth embodiment;

FIG. 22 is a view illustrating a state in which the endoscope of FIG. 21 having a transparent cap mounted thereon approaches the lumen wall;

FIG. 23 is a view illustrating a state in which the lumen wall is pinched;

FIG. 24 is a view illustrating a state in which a high-frequency surgical knife is pierced into a lumen wall while the lumen wall is pinched;

FIG. 25 is a view illustrating a state in which an opening treating device is inserted into the inside of an abdominal cavity through an opening in the lumen wall formed in FIG. 24;

FIG. 26 is a view showing a state in which an opening in the lumen wall formed in FIG. 24 is dilated by a balloon dilator;

FIG. 27 is an external view showing an entire system according to a fifth embodiment;

FIG. 28 is a view illustrating a state in which a high-frequency surgical knife is pierced into a lumen wall while the lumen wall is pinched;

FIG. 29 is a view illustrating a state in which an endoscope of a system according to a sixth embodiment is opposed to a lumen wall;

FIG. 30 is a view showing a state in which a lumen wall is tied and electrically excised by means of a high-frequency snare of the system according to the sixth embodiment;

FIG. 31 is a view illustrating a state in which an opening is formed in a lumen wall by means of excision using the snare of FIG. 30;

FIG. 32 is an entire view showing a high-frequency snare in the system according to the sixth embodiment;

FIG. 33 is an external view showing an entire system according to a seventh embodiment;

FIG. 34 is a view illustrating a treatment system according to prior art;

FIG. 35 is a schematic view showing an endoscopic treatment device according to prior art, the treatment device being formed as a balloon catheter;

FIG. 36 is a view illustrating another endoscopic treatment device according to prior art;

FIG. 37 is a schematic view showing a high-frequency catheter according to prior art; and

FIG. 38 is a schematic view showing another high-frequency catheter according to prior art.

### DETAILED DESCRIPTION OF THE INVENTION

### First Embodiment

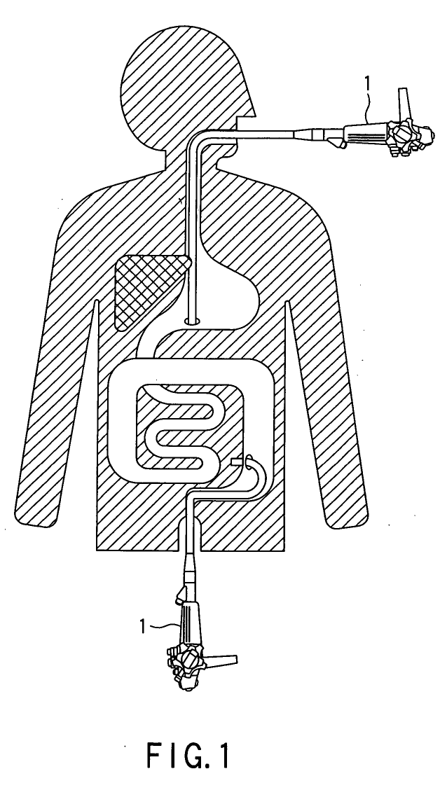

FIG. 1 to FIG. 10 each show an endoscopic system for treating the inside of a body cavity according to a first embodiment of the present invention.

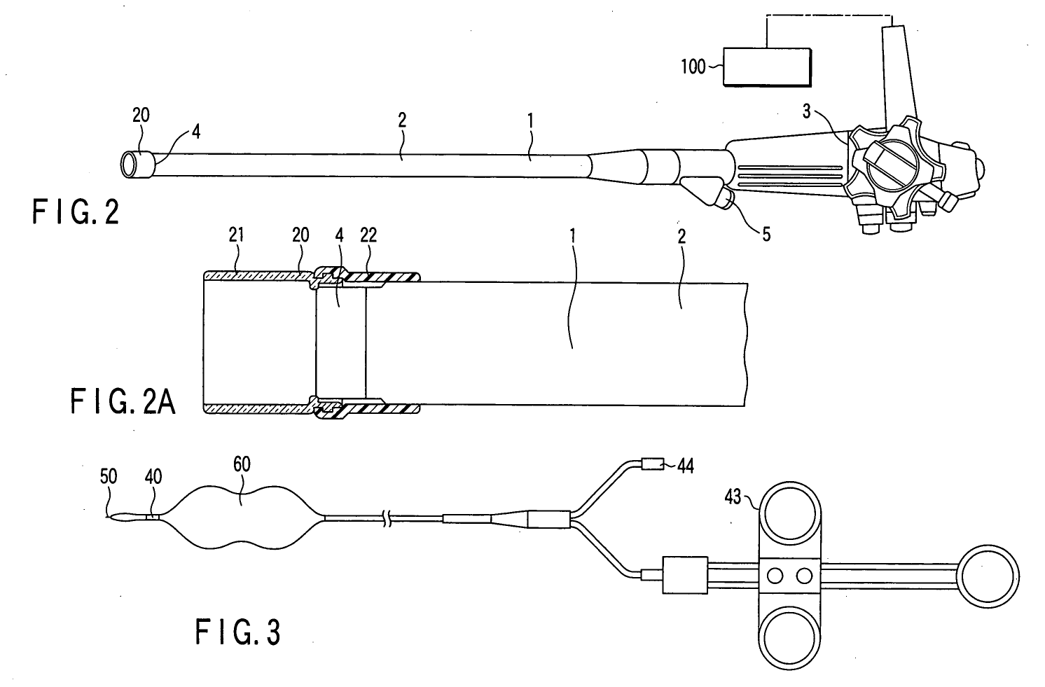

As shown in FIG. 1 and FIG. 2, an endoscopic system for treating the inside of a body cavity according to the present embodiment comprises the endoscope 1 inserted into a body through a natural opening of a human body. Further, a system according to the present embodiment comprises: a transparent cap 20 mounted on a distal end portion of an endoscope 1 as shown in FIG. 2A; and an opening treatment device 40 inserted into a body via the endoscope 1 as shown in FIG. 3.

This endoscope 1 comprises an endoscopic insert portion 2 to be inserted into a body; an endoscope distal end portion 4 that is at a distal end of an endoscope insert portion; and an endoscope manipulating portion 3 for manipulating an endoscope insert portion. This endoscope manipulating portion 3 is connected to an endoscope main body or a suction unit 100 via a universal cable or the like (not shown). A forceps opening 5 provided at this endoscope manipulating portion 3 communicates with a channel formed of a tubular member (not shown) extending in the insert portion 2 to the distal end portion 4. The length of the endoscope insert portion 2 is 300 mm to 5000 mm to an extent such that the insert portion can be inserted into a body through a natural opening. In particular, it is preferable that the length be 1000 mm to 2000 mm. The outer diameter is 3 mm to 30 mm to an extent such that the insert portion can be inserted through a natural opening of a human body. In particular, it is preferable that the outer diameter be 3 mm to 25 mm.

As shown in FIG. 2A in an enlarged manner, a transparent cap 20 comprises a hood 22 that is a tubular member press-fitted to the endoscope distal end portion 4; and a cylinder member 21 securely fitted to a distal end of the hood 22.

It is preferable that the cylinder member 21 be made of a transparent polymeric resin material (such as acrylic resin, polystyrene, polycarbonate, polyethylene terephthalate, polyethylene naphthalate, and so on). The outer diameter of this cylinder member 21 is 3 mm to 30 mm to an extent such that the cylinder member can be inserted through a natural opening of a human body, and is preferably 5 mm to 20 mm. The length is 0.5 mm to 30 mm, and is preferably 3 mm to 20 mm.

The hood 22 is preferably formed of an expandable polymeric resin material (such as vinyl chloride, vinyl chloride-vinyl acetate copolymer, polyurethane, fluorine resin, and so on), or an elastic material (synthetic rubbers such as natural or synthetic rubber latex, silicone rubber, isoprene rubber, neoprene rubber, or elastomers including, as a main component, polystyrene, polyester, polyether, etc.). This hood is configured to be removable without damaging the endoscope distal end portion 4. This hood 22 is fixed to the cylinder member 21 by suitable means such as press-fit, adhesive, ultrasonic fusion, thermal fusion, or solvent adhesive.

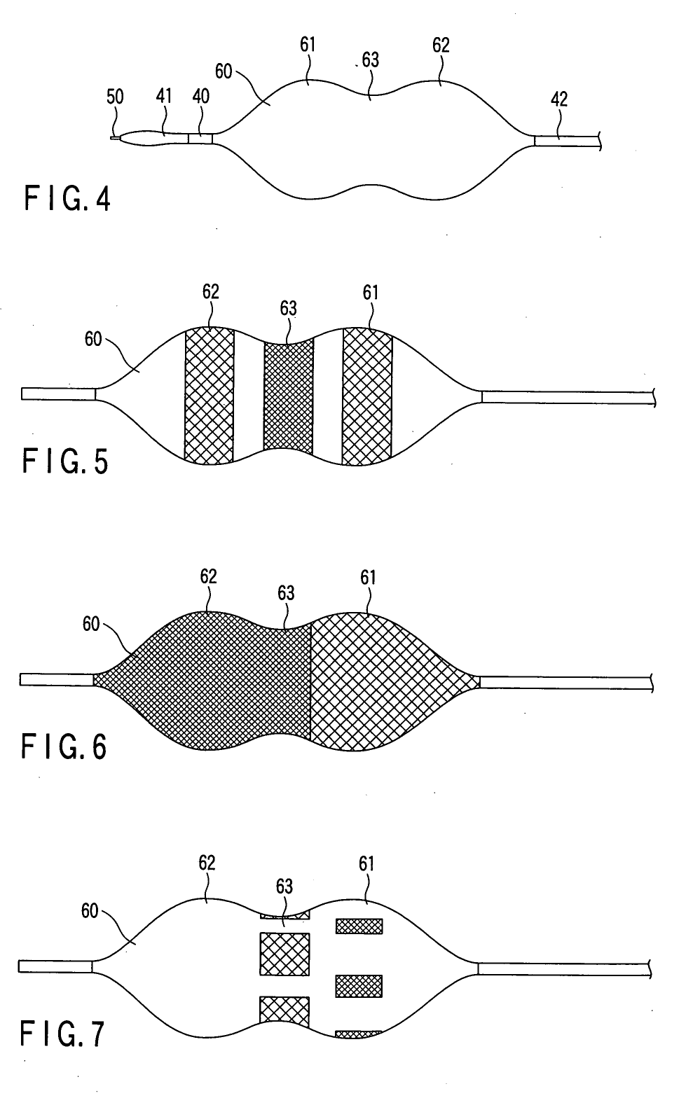

As shown in FIG. 3 and FIG. 4, the opening treatment device 40 is composed of: a sheath 42; a high-frequency surgical knife 50 inserted into this sheath; a balloon dilator 60 securely fitted to the outer periphery portion of the sheath 42; a surgical knife manipulating portion 43 for manipulating the high-frequency surgical knife 50; and a water supply port 44.

The sheath 42 of this opening treatment device 40 has a hollow structure whose cross section is circular, for example. This sheath is preferably formed of an insulation polymeric resin material (such as synthetic polymeric polyamide, high density/low density polyethlene, polyester, polytetrafluoroethylene, tetrafluorotethylene-perfluoro alkyl vinyl copolymer, tetrafluoro ethylene-hexafluoro propylene copolymer, and so on). At least two lumens are extended inside of this sheath 42, and one of these lumens can insert the high-frequency surgical knife 50. The other can route a fluid supplied from the water supply port 44, for example, in order to dilate a balloon dilator 60.

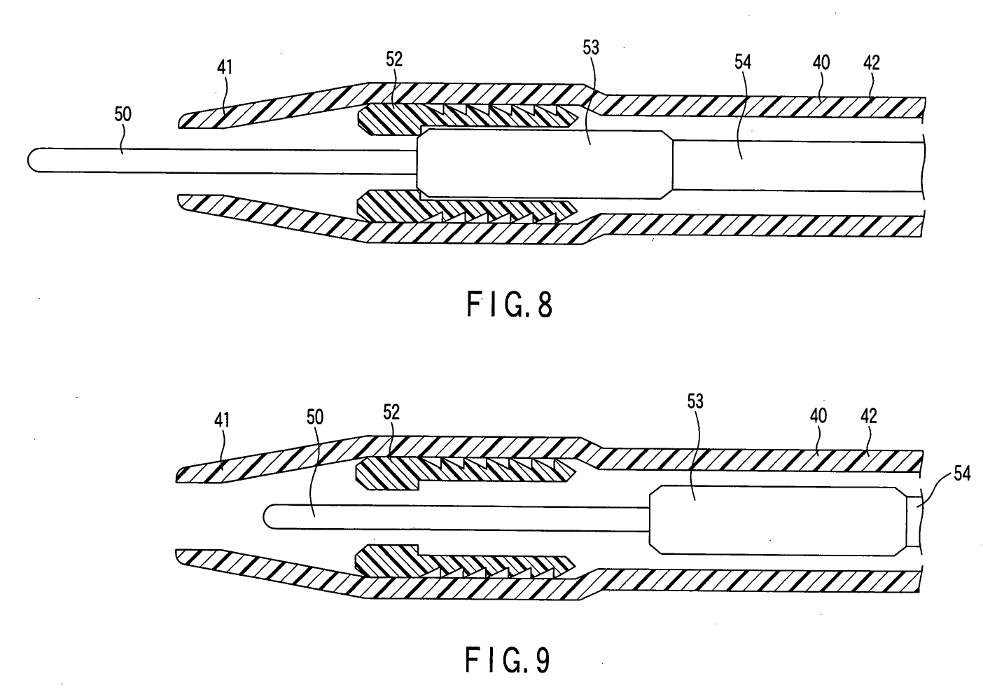

As shown in FIG. 8 to FIG. 10 in an enlarged manner, the opening treatment device 40 has a distal end portion 41 in a tapered shape. The sheath 42 has a portion that is slightly dilated in diameter at a site adjacent to this distal end portion 41. Then, a substantially cylindrical stopper 52 having a stepped internal hole formed to restrict movement of the high-frequency surgical knife 50 is housed in this dilated diameter portion.

The high-frequency surgical knife 50 is securely fitted to a wire 54 at its proximal end of a surgical knife manipulating portion 43 via a wire side stopper 53. This wire 54 is mounted removably on the surgical knife manipulating portion 43 at its proximal end. A high-frequency power source (not shown) is connected to the surgical knife manipulating portion 43 via a high-frequency cable (not shown).

The high-frequency surgical knife 50 is made of an electrically conductive metal (such as stainless steel), and its cross section may have a circular or paddle shape. When the surgical knife is formed to have a circular cross section, it is preferable that the outer diameter be 0.1 mm to 10 mm. In particular, it is preferable that the outer diameter be 0.3 mm to 1.0 mm. When the surgical knife is formed to have a paddle shaped cross section, it is preferable that the surgical knife be formed in a substantially rectangular shape of 0.2 mm to 1.0 mm in length of one side, and in particular, of 0.2 mm to 0.5 mm×0.5 mm to 1.0 mm in side length.

The wire side stopper 53 has a substantially tubular structure made of an electrically conductive metal (such as stainless steel), and the length is 1 mm to 20 mm. In particular, it is preferable that the length be 3 mm to 10 mm. In addition, this wire side stopper 53 has its outer diameter that is smaller than the internal hole of the larger diameter of the sheath side stopper 52 and that is larger than the internal hole of the smaller diameter.

The wire 54 is formed of, for example, a single or twisted wire of electrically conductive metallic (such as a stainless steel). It is preferable that the sectional shape of this wire 54 be circular. The outer diameter of this wire 54 is 0.1 mm to 15 mm such that the wire can be inserted into the sheath 42. In particular, it is preferable that the outer diameter be 0.3 mm to 3 mm. The length of this wire 54 is 300 mm to 5000 mm. In particular, it is preferable that the outer diameter be from 1000 mm to 2000 mm.

The high-frequency surgical knife 50 can protrude from a distal end of the sheath 42. When the high-frequency surgical knife 50 is inserted into the sheath 42, the wire side stopper 53 securely fitted to the high-frequency surgical knife 50 abuts against a stepped portion formed in an internal hole of the sheath side stopper 52. In this manner, the high-frequency surgical knife 50 is restricted from sliding in a further distal end direction. It is preferable that the sheath side stopper 52 be formed to allow a member having no stopper, such as a guide wire 45 for guiding the sheath 42 to a target site, to be freely inserted into the stopper 52. Thus, it is preferable that a small diameter portion of the internal hole of the system side stopper 52 and the inner diameter of the distal end portion 41 be formed to be slightly larger than the size of the guide wire 45 and so on.

As shown in FIG. 4, a balloon dilator 60 is provided as a balloon made of a polymeric resin material. This dilator has a distally maximal outer diameter portion 61; a center portion 63; and a proximally maximal outer diameter portion 62. The distally maximal outer diameter 61 and proximally maximal outer diameter portion 62 are each have a larger outer diameter that that of the center portion 63 when the balloon dilator 60 is dilated. It is preferable that the center portion 63 has the outside diameter sufficient to form the opening to such an extend that the inner diameter of the opening is in the range of 3 mm to 100 mm, and especially of 5 mm to 30 mm appropriate to allow the endoscope 1 and the transparent cap 20, and additionally an after-mentioned over-tube when combined, to be inserted into the opening. The distally maximal outer diameter portion 61 and proximally maximal outer diameter portion 62 are formed one size larger than the outer diameter of the center portion 63 in order to prevent deviation in use. For example, the outer diameter is 5 mm to 120 mm, for example. In particular, it is preferable that the outer diameter be formed to be about 7 mm to 50 mm. This balloon dilator 60 can be dilated due to a pressure of a fluid supplied from an inflation device (not shown) removably connected to a water supply port 44 at the proximal end of the opening treatment device sheath 42.

This balloon dilator 60 can be easily positioned by applying suitable markings as shown in FIG. 5 to FIG. 7, for example. The balloon dilator 60 shown in FIG. 5 has a ring shaped marking for the distally maximal outer diameter portion 61, the center portion 63, and the proximally maximal outer diameter 62, respectively. The balloon dilator 60 shown in FIG. 6 has a marking formed in an entirely coloring pattern so as to identify a position of the center portion 63. In addition, the balloon dilator 60 shown in FIG. 7 has a plurality of markings with their different sizes or colors at the center portion 63 and proximally maximal outer diameter portion 62. The features of these markings can be combined properly according to their uses. Such markings of the balloon dilator 60 having the distally maximal outer diameter portion 61, center portion 63, and proximally maximal outer diameter portion 62 are useful even in any other general treatment without being limited to this surgical operation described later. Further, these markings use or mix a radiopaque materials such as tungsten, platinum, barium sulfate, bismuth oxide, thereby enabling checks under X-ray fluoroscopy.

Now, an exemplary surgical operation using an endoscopic system for treating the inside of a body cavity according to the present embodiment will be described with reference to FIG. 11 to FIG. 16.

First, the endoscope 1 having the transparent cap 20 mounted thereon is inserted through natural openings of a human body, that is, nose, mouth, anus, or vagina into lumens in the body, that is, esophagus, stomach, duodenum, small intestine, large intestine, rectum, vagina, uterus, etc.. The inside of a body cavity is observed through this endoscope 1. The distal end portion 4 and transparent cap 20 are opposed to a target site of a lumen wall 101, and is disposed in a state shown in FIG. 11.

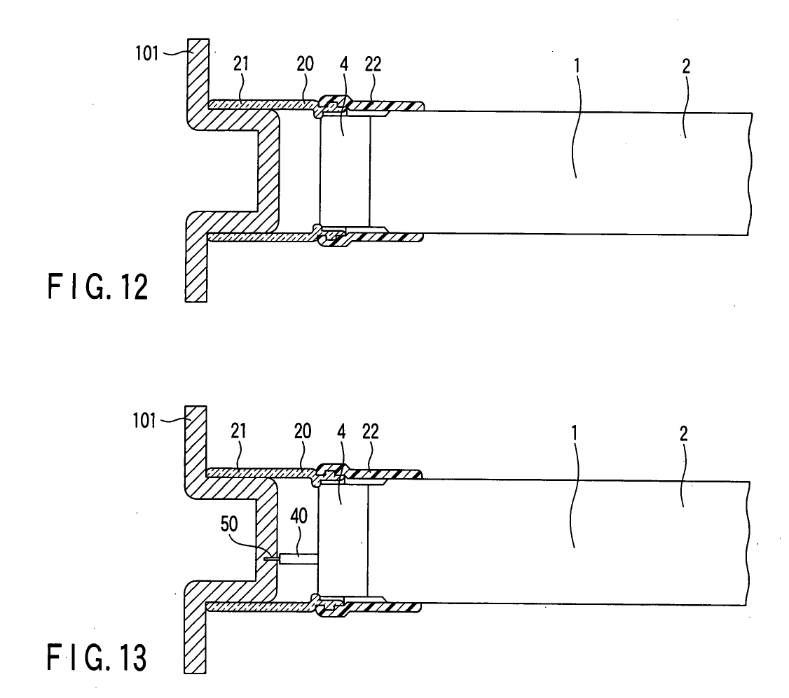

Next, the transparent cap 20 is abutted against a target site of the lumen wall 101, and the lumen wall 101 is suctioned by a suctioning machine (not shown). The lumen wall 101 is suctioned into the transparent cap 20, and a recess is formed, as shown in FIG. 12.

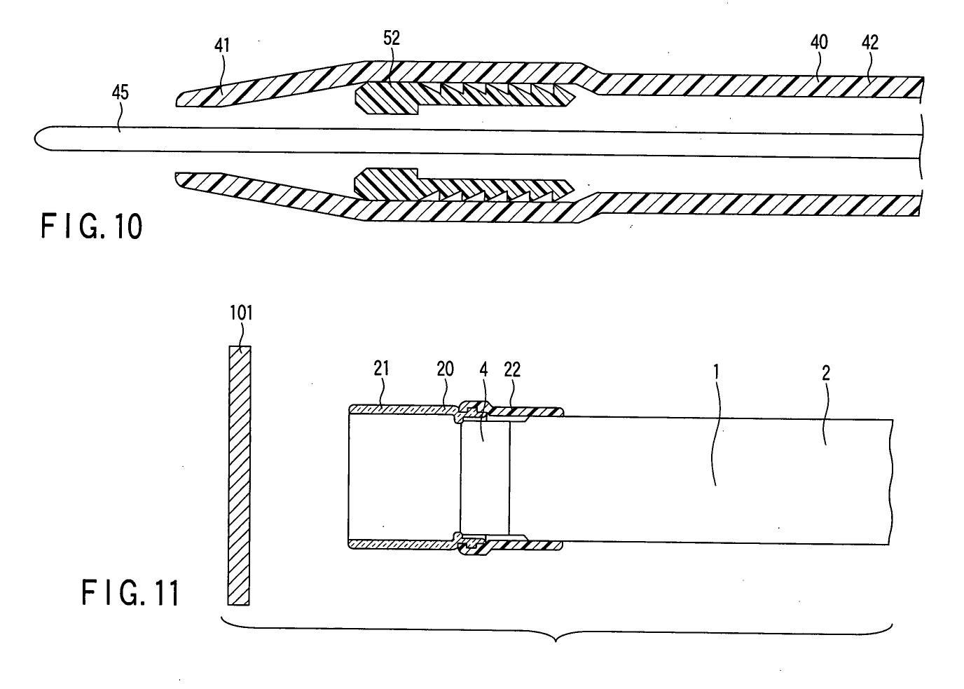

In this state, the opening treatment device 40 shown in FIG. 3 is inserted from the forceps 5 (FIG. 2) of the endoscope 1 into a channel (not shown) arranged in the insert portion 2, and a distal end portion 41 of this treatment device 40 is protruded from the endoscope distal end portion 4. At this time, the high-frequency surgical knife 50 is retracted from the distal end face of the opening treatment device 40 by manipulation of the surgical knife manipulating portion 43 so as not to damage a channel in the insert portion 2 (a state shown in FIG. 9). Then, after the opening treatment device 40 has been made to protrude from the endoscope distal end portion 4, the high-frequency surgical knife 50 is protruded from the distal end face of the opening treatment device 40 by manipulation of the surgical knife manipulating portion 43 (a state shown in FIG. 8). Then, the surgical knife is abutted against a lumen wall 101 having a recess formed thereon. In this state, a high-frequency current is supplied from a high-frequency power source (not shown) to a high-frequency knife 50, and the lumen wall 101 is perforated, as shown in FIG. 13.

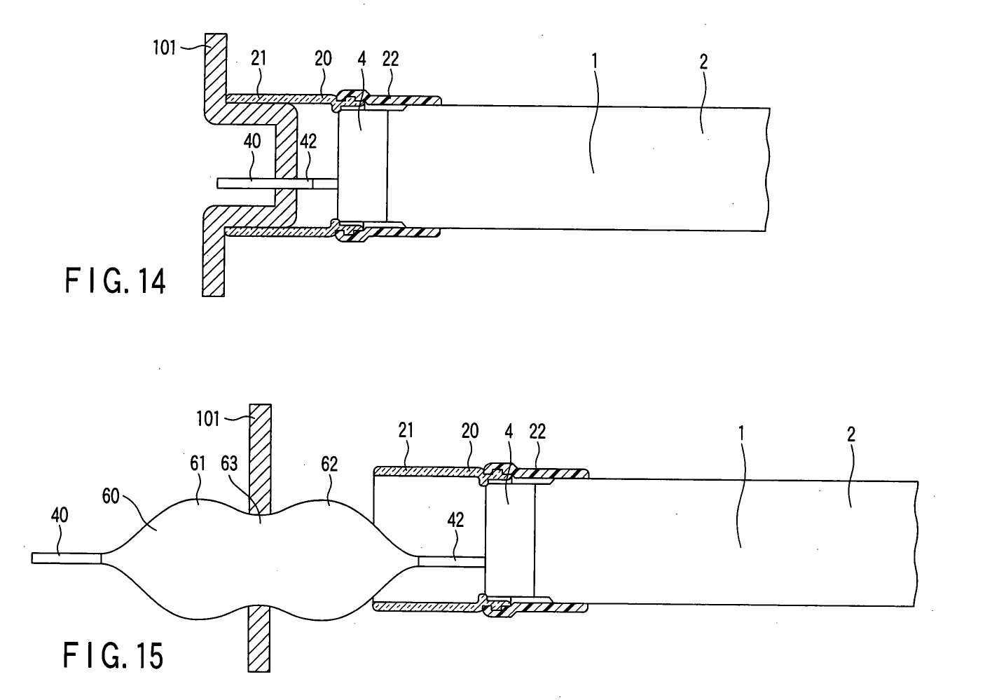

After the lumen wall 101 has been perforated, the opening treatment device 40 is made to further protrude, and the opening treatment device sheath 42 is inserted into the outside of the lumen wall, i.e., thoracic cavity or abdominal cavity and the like. This state is shown in FIG. 14. After the center portion 63 of the balloon dilator 60 arranged at the outer periphery portion of the opening treatment device sheath 42 is inserted until abutted against the lumen wall 101, a fluid is supplied to the balloon dilator 60 by means of an inflation device (not shown). Then, this balloon dilator 60 is dilated, as shown in FIG. 15. After the balloon dilator 60 has been dilated up to the size such that an opening of the lumen wall 101 can accept the endoscope 1, the supply of fluid is stopped.

After sufficient dilation of the balloon dilator 60 has been completed, the cap 20 and distal end portion 4 of the endoscope 1 are inserted into the lumen wall 101, and is made to protrude to the outside of the lumen wall, i.e., to the inside such as abdominal cavity or thoracic cavity. The opening treatment device 40 is withdrawn from the forceps 5, thereby observing or treating of the outside of the lumen wall, i.e., abdominal cavity or thoracic cavity.

According to the endoscopic system for treating the inside of a body cavity of the present embodiment, the lumen wall 101 is suctioned by using the transparent cap 20, and a recess is produced. In this manner, a target site can be fixed, and the target site can be reliably perforated. In addition, another organ adjacent to the outside of the lumen wall 101 can be spaced from a dissection site when the high-frequency surgical knife 50 perforates the lumen wall.

The high-frequency wire 54 and balloon dilator 60 are arranged at the opening treatment device 40, whereby the perforating and dilation of the lumen wall 101 can be continuously carried out, and simplified surgical operation and time reduction can be expected.

The balloon dilator 60 has the distally maximal outer diameter portion 61 and proximally maximal outer diameter portion 62 that are larger than the center portion 63 at both end sides of the center portion 63. This makes it possible to prevent displacement of the balloon dilator 60 being dilated.

In addition, a marking formed at the balloon dilator 60 makes it possible to facilitate positioning of the opening treatment device 60 relevant to the lumen wall 101 of the balloon dilator 60 of the opening treatment device 40.

### Second Embodiment

FIG. 17 and FIG. 18 show a second embodiment of the present invention. In a variety of embodiments described below, like elements similar to those according to the first embodiment are designated by like reference numerals. A detailed description is omitted here.

An endoscopic system for treating the inside of a body cavity according to the present embodiment is composed of an over-tube 30, an endoscope 1 inserted into this over-tube 30; and an opening treatment device 40 inserted into this endoscope 1.

The over-tube 30 consists of a tubular over-tube sheath 31; and a proximal portion 32 disposed at a proximal end of the over-tube sheath 31. A suction port 33 communicating with the inside of the over-tube 30 is provided at this proximal portion 32.

The over-tube sheath 31 has a hollow structure whose cross section is circular, for example, and is formed of a polymeric resin material such as polytetrafluoroethylene (PTFE), expanded polytetrafluoroethylene (ePTFE), polyurethane, styrene series elastomer, olefin series elastomer, and silicone. This sheath has at least one lumen for inserting the endoscope 1. The length is 300 mm to 5000 mm to an extent such that the sheath can be inserted through a natural opening of a human body, and can reach a target site. In particular, it is preferable that the length be 1000 mm to 2000 mm. The outer diameter is 3 mm to 30 mm to an extent such that the sheath can be inserted through a natural opening of a human body. In particular, it is preferable that the outer diameter be 3 mm to 25 mm. The inner diameter is 3 mm to 30 mm to an extent such that the endoscope 1 can be inserted. In particular, it is preferable that the inner diameter be 3 mm to 25 mm.

A proximal portion 32 of the over-tube 30 is formed of a hard pipe shaped member. This portion is fixed at an end portion of the over-tube sheath 31, by suitable means such as press-fit, adhesive, ultrasonic wave fusion, thermal fusion, solvent adhesive, screws and so on. In order to efficiently make a suction operation or air supply operation via the endoscope 1, a valve (not shown) is disposed at the over-tube proximal portion 32. The air tightness in the body against the outside of the body is maintained by the valve.

A suction port 33 connects a suction tube (not shown) connected to a suction machine 100, for example, via a tubular member. Alternatively, a cap (not shown) can be mounted in order to hold the inside of the body.

FIG. 19 illustrates a surgical operation using a system according to the second embodiment.

First, the endoscope 1 inserted into the over-tube 30 is inserted through natural openings of a human body, that is, nose, mouth, anus, or vagina into lumens in the body, that is, esophagus, stomach, duodenum, small intestine, large intestine, rectum, vagina, uterus, etc..

Next, the over-tube 30 is abutted against the lumen wall 101, and the lumen wall 101 is suctioned by means of a suctioning machine 100, whereby the lumen wall 101 is suctioned in the sheath 31 of the over-tube 30, and a recess is formed. At this time, a suction function of the endoscope 1 may be used, and a suction tube (not shown) may be connected to a suction port 33 of the over-tube 30, thereby carrying out suctioning operation.

According to the system of the second embodiment, a larger recess can be obtained by means of suctioning operation using the over-tube 30. That is, a suction tube (not shown) is connected to the suction port 3 of the over-tube 30, whereby a passage having a larger sectional area as compared with the channel of the endoscope 1 is formed even when the endoscope 1 is inserted. Thus, a section operation can be carried out through the passage or inner diameter in the over-tube 30 with its small channel resistance, and a larger recess can be formed within a short time.

### Third Embodiment

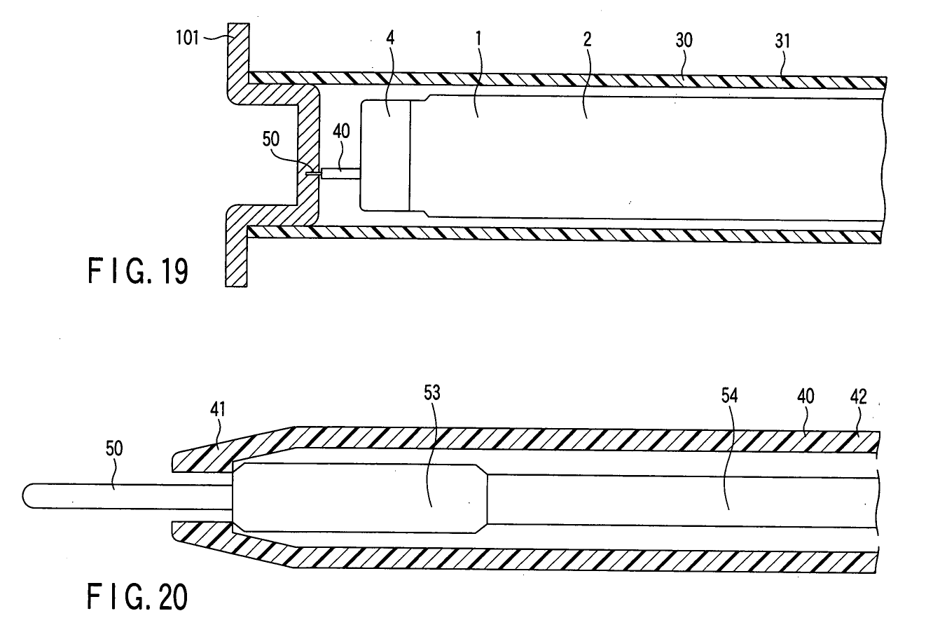

FIG. 20 shows a distal end portion 41 of an opening treatment device 40 according to the system of a third embodiment of the present invention.

A high-frequency surgical knife 50 according to the present embodiment can be removed from an opening treatment device sheath 42. This sheath 42 is reduced in inner diameter of a distal end portion 41 as compared with that of the above-described embodiment, and a shoulder portion is formed. A diameter dilution portion for housing a sheath side stopper in the above-described embodiment is omitted here. When the high-frequency surgical knife 50 is inserted into the opening treatment device sheath 42, a wire side stopper 53 securely fitted to the high-frequency knife 50 abuts against a shoulder portion formed in inner hole of the distal end portion 41. In this manner, the sliding of the high-frequency knife 50 to the distal end direction is restricted.

In this embodiment, the sheath 42 itself restricts the sliding of the high-frequency knife 50 to the distal end direction so that the distal end portion of the high-frequency knife 50 is not made to protrude excessively from the sheath 42. Thus, the number of parts is reduced as compared with that in the above-described embodiment. Accordingly, the structure of the system is simplified and cost reduction can be expected.

### Fourth Embodiment

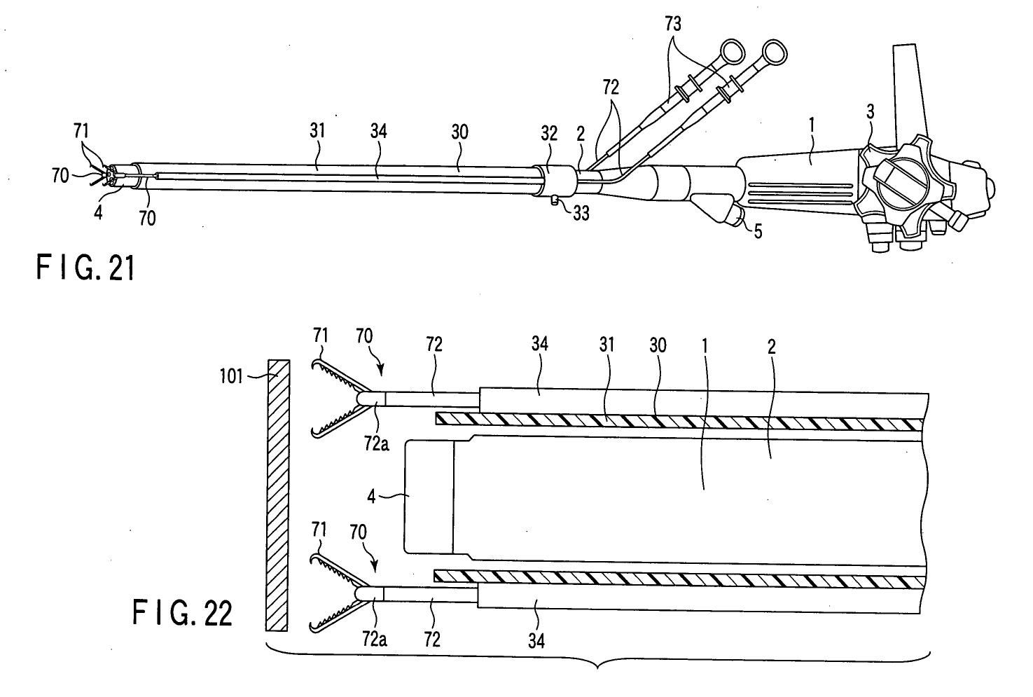

FIG. 21 to FIG. 26 each show a system according to a fourth embodiment of the present invention.

As entirely shown in FIG. 21, a system according to the present embodiment is composed of an over-tube 30; an endoscope 1 inserted into the over-tube; a channel member 34 arranged along an outside portion of the over-tube 30; a grasping forceps 70 inserted into this channel member 34; and an opening treatment device 40 inserted into the endoscope 1. The opening treatment device 40 may be identical to that described in the above-described embodiment, and is not illustrated in FIG. 21.

A channel 34 arranged in the over-tube 30 has a tube structure whose cross section is circular. This channel is made of a polymeric resin material similar to the above-described sheath 42 or the over-tube sheath 31, for example, and has at least one bore or lumen for passing the grasping forceps 70. This channel member 34 may be integrally formed at the over-tube 30, for example, or alternatively, may be formed as another member capable of being fitted to the outside. When the channel member is fixed to the outside, it can be fixed to the over-tube sheath 31 by using suitable means such as press-fit, adhesive, ultrasonic wave fusion, thermal fusion, solvent adhesive, screws, etc.. The length is 300 mm to 5000 mm to an extent such that the channel member can be inserted into the body together with the over-tube. In particular, it is preferable that the length be 1000 mm to 2000 mm. The inner diameter is 1 mm to 20 mm to an extent such that the grasping forceps can pass. In particular, it is preferable that the inner diameter be 2 to 10 mm.

The grasping forceps 70 comprises a grasping forceps sheath 72 inserted into a channel member 34; a hollow distal end cover 72a located at a distal end of the grasping forceps sheath 72; and a pair of forceps members 71 rotatably supported on this distal end cover, as shown in FIG. 22. A proximal end of this forceps member 71 is connected to a manipulating wire (not shown) inserted into the pinch forces sheath 72. Then, this proximal end is manipulated to be opened/closed by a grasping forceps manipulating portion 73 (refer to FIG. 21) securely fixed to the proximal end of the grasping forceps sheath 72.

The grasping forceps sheath 72 has an irregular coil on the internal and external circumference face on which a metallic wire (such as a stainless steel wire) with its circular cross section is closely wound. At this sheath 72, a polymeric resin material based coating similar to the above-described sheath 42 may be applied to the outer periphery portion of the coil. The sheath 72 is, therefor, formed into a structure which is prevented from buckling even if a force in a compressing direction is applied on the distal end portion and the proximal end portion.

In addition, the grasping forceps sheath 72 can be formed of a metallic wire (such as a stainless steel wire) which has rectangular cross section deformed from circular shape, for example. Thus, this sheath can be formed by closely winding a wire having this rectangular cross section. In this case, a coil sheath with its flat internal and external faces is formed, and the manipulating wire can be easily actuated. In addition, even if the element wire of the same element wire diameter are used, a coil sheath having a large internal diameter can be obtained as compared with a round shaped coil sheath. This makes it further easier to operate the manipulating wire.

Further, the grasping forceps sheath 72 may be a tube sheath made of a polymeric resin material similar to the sheath 42, for example. In this case, the internal and external faces of the sheath have a slipping property, thus facilitating insertion into, or removal from the endoscope channel and actuating the manipulating wire. Further, the outer face of the grasping forceps sheath 72 made of a polymeric resin material may be processed by embossing. A frictional resistance relevant to the inner peripheral face of the endoscope channel is lowered by emboss processing, and insertion and removal can be facilitated.

In addition, the grasping forceps sheath 72 may be formed as a double-layered tube having an internal layer and an external layer at a wall portion, and further, a reinforced member may be embedded between these internal and external layers. In this case, it is preferable that the internal layer and external layer be formed of the polymeric resin material. In this manner, even when the force in the compression direction acts with the distal end portion and proximal end portion of the sheath 72, the compression resistance is much better as compared with a tube sheath without embedding a reinforced member therein, and sheath buckling is prevented.

The outer diameter of the grasping forceps sheath 72 is such that the sheath can be inserted into the channel 34. The thickness of the sheath is determined depending on rigidity of the element material. It is preferable that the thickness when the sheath 72 be formed of a metal material, for example, is about 0.2 mm to 0.5 mm, and the thickness when the sheath is made of a polymeric resin material be about 0.3 mm to 0.6 mm. In addition, when a reinforced member is embedded, there is an advantage that the thickness of the sheath made of a polymeric material is decreased, and the inner diameter can be increased.

The manipulating wire is provided as a wire made of a metal material (such as stainless steel), for example, and formed of a single wire or a twisted wire. It is preferable that the cross section of this manipulating wire be formed in a circular shape. The outer diameter is 0.1 mm to 5 mm. In particular, it is preferable that the outer diameter be 0.3 mm to 1 mm. The length is 300 mm to 5000 mm. In particular, it is preferable that the length be 1000 mm to 2000 mm.

The manipulating wire may be coated with the polymeric resin, and the sliding properties of the manipulating wire can be improved. In this case, the coating thickness is about 0.05 mm to 0.3 mm.

In addition, the manipulating wire may be inserted into a thin tube made of a polymeric resin similar to the sheath 42, for example. In this case as well, the sliding properties can be improved. It is preferable that the thickness of the tube be about 0.05 mm to 0.3 mm.

Now, a surgical operation using a system according to a fourth embodiment will be described with reference to FIG. 22 to FIG. 26.

First, the endoscope 1 penetrating through the outer-tube 30 is inserted through natural openings of a human body, that is, nose, mouth, anus, or vagina into lumens in the body, that is, esophagus, stomach, duodenum, small intestine, large intestine, rectum, vagina, uterus, etc..

Next, a grasping forceps 70 is inserted into a channel member 34 extended along the over-tube 30. After a forceps member 71 has been made to protrude from the distal end portion of the channel member 34, a forceps manipulating portion 73 is manipulated. Then, the forceps member 71 is opened as shown in FIG. 22.

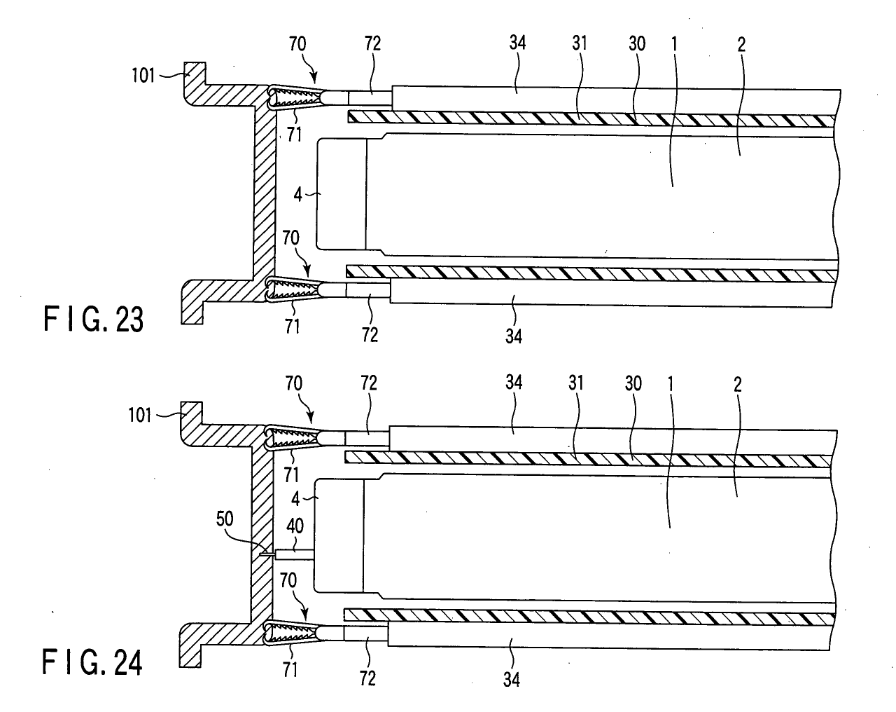

Next, the forceps member 71 is abutted against the lumen wall 101, the forceps manipulating portion 73 is manipulated, and the forceps member 71 is closed. Then, the grasping forceps 70 having the lumen wall 101 pinched thereon or the over-tube 30 is pulled proximally, i.e., toward the manipulating portion 3, and a recess as shown in FIG. 23 is formed.

In this state, the opening treatment device 40 is inserted through the forceps opening 5 of the endoscope 1. Then, the opening treatment device is made to protrude from the endoscope distal end portion 4 through a channel arranged in the endoscope insertion portion 2 (not shown). At this time, the high-frequency surgical knife 50 is inserted while the knife is retracted from the distal end face of the opening treatment device 40 by manipulating the surgical knife manipulating portion 43 so as not to damage the channel. While the opening treatment device 40 is protruding from the opening treatment device 4, the high-frequency surgical knife 50 is made to protrude from the distal end face of the opening treatment device 40 by manipulating the surgical knife manipulating portion 43. Then, the protruding surgical knife 50 abuts against a lumen wall 101 having a recess formed thereon. Then, a high-frequency current is supplied by means of a high-frequency power source (not shown), and the lumen wall 101 is perforated, as shown in FIG. 24.

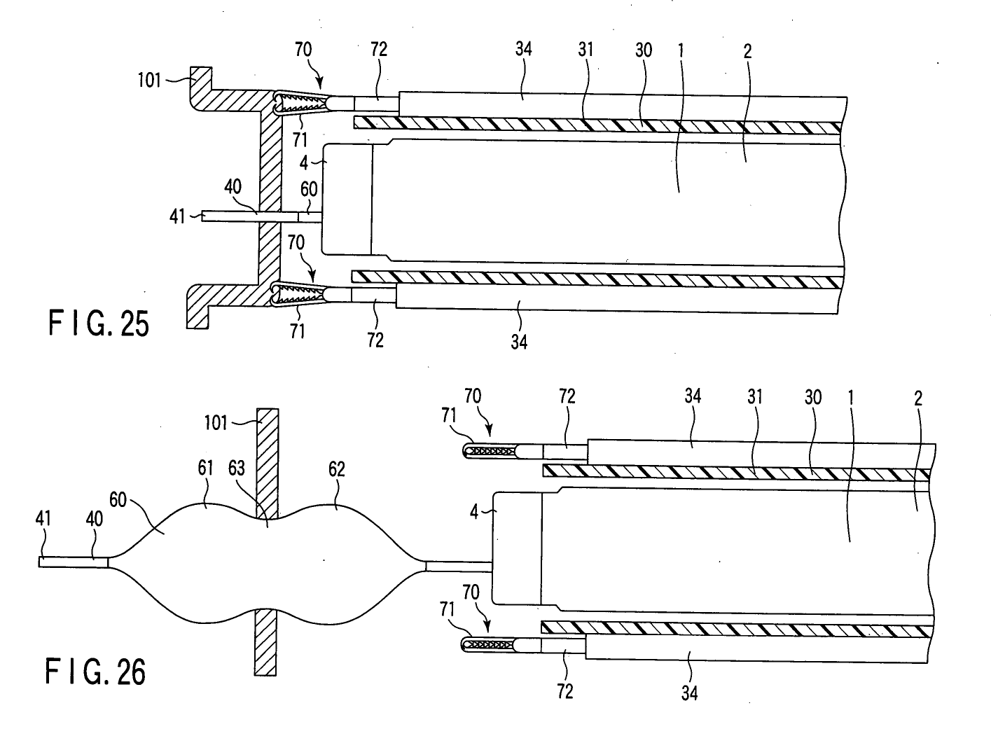

After the lumen wall 101 has been perforated, the opening treatment device 40 is made to further protrude from the distal end portion of the endoscope 1, and then, the opening treatment device sheath 42 is inserted into the outside of the lumen wall, i.e., a thoracic cavity or abdominal cavity, as shown in FIG. 25. At this time, it is desirable that the high-frequency surgical knife 50 be retracted into the opening treatment device sheath 42. After a center portion 63 of a balloon dilator 60 arranged at the opening treatment device sheath 42 is inserted until the center portion 63 has been abutted against the lumen wall 101, the forceps manipulating portion 73 is manipulated, and the lumen wall 101 is released. Then, a fluid is supplied to the balloon dilator 60 by means of the balloon manipulating portion, and an opening of the lumen wall 101 is dilated to a size sufficient to insert the endoscope 1, as shown in FIG. 26.

When sufficient dilation is obtained, the endoscope 1 is inserted into the outside of a lumen wall, i.e., abdominal cavity or thoracic cavity, the opening treatment device 40 is withdrawn from the forceps opening 5, thereby observing or treating the outside of a lumen wall, i.e., abdominal cavity or thoracic cavity.

According to a system of the fourth embodiment, a recess is produced on the lumen wall 101 by using a forceps 70, whereby a target site can be perforated reliably and easily while the target site is fixed. In addition, another organ adjacent to the outside of the lumen wall 101 can be spaced from the perforating area during perforating the high-frequency knife 50. Therefore, an opening having its required size can be formed safely and reliably at a target site of the lumen wall 101 within a short time.

### Fifth Embodiment

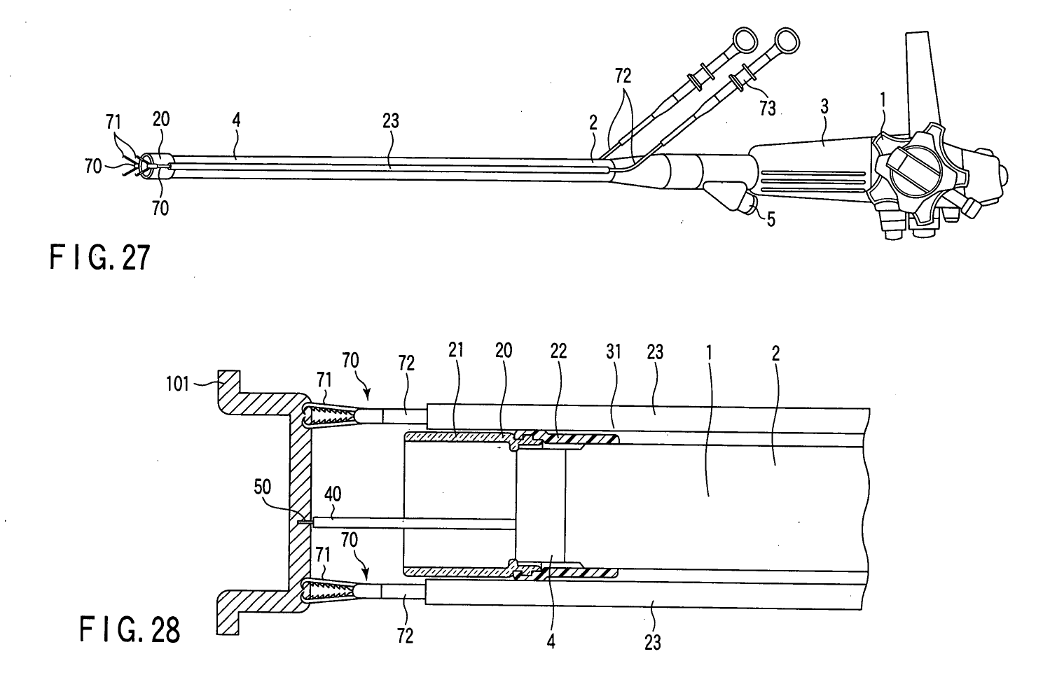

FIG. 27 and FIG. 28 each show a fifth embodiment of the present invention.

A system according to the present embodiment is composed of an endoscope 1; a transparent cap 20 mounted on an endoscope distal end portion 4; a channel member 23 having its distal end fixed to the transparent cap 20; a grasping forceps 70 inserted into the channel member 23; and an opening treatment device 40 inserted into the endoscope 1.

The channel member 23 having its distal end fixed to the transparent cap 20 is formed of a tube whose cross section is circular. This member is formed of a polymeric resin material similar to the sheath 42 or the over-tube sheath 31, for example, and has at least one lumen for inserting the grasping forceps 70. The distal end of this channel member 23 may be molded integrally with the transparent cap 20, for example. Alternatively, this distal end may be formed of another member or may be fixed to this transparent cap 20. When the distal end is fixed, it can be fixed to the transparent cap 20 by suitable means such as press-fit, adhesive, ultrasonic wave fusion, thermal fusion, solvent adhesive, or screws and the like.

A length of this channel member 23 is 300 mm to 5000 mm to an extent such that the channel member can be inserted into a body together with the endoscope 1. In particular, it is preferable that the length be 1000 mm to 2000 mm. The inner diameter is 1 mm to 20 mm to an extent such that the grasping forceps passes. In particular, it is preferable that the inner diameter be 2 mm to 10 mm.

FIG. 28 shows a state in which an opening is formed on the lumen wall 101 by using a system according to the fifth embodiment.

First, the endoscope 1 having the transparent cap 20 mounted thereon is inserted through natural openings of a human body, that is, nose, mouth, anus, vagina, etc. into lumens in the body, that is, esophagus, stomach, duodenum, small intestine, large intestine, rectum, vagina, uterus, etc..

Next, the grasping forceps 70 is inserted into the channel member 23 fixed to the transparent cap 20, and a distal end portion of the forceps member 71 is made to protrude from the distal end portion of the channel member 23. Then, the forceps manipulating portion 73 is manipulated, and the forceps member 71 is opened.

Next, after the forceps member 71 has been abutted against the lumen wall 101, the forceps manipulating portion 73 is manipulated, whereby the forceps member 71 is closed, and the lumen wall 101 is pinched. Then, the grasping forceps 70 or transparent cap 20 is pulled toward the proximal side, whereby a recess is formed on the lumen wall 101.

In this state, the opening treatment device 40 is inserted through the forceps opening 5 of the endoscope 1. Then, the opening treatment device 40 is made to protrude from the endoscope distal end portion 4 through a channel arranged in the endoscope insert portion 2 (not shown). At this time, it is desirable that the high-frequency surgical knife 50 be inserted while the knife is retracted from the distal end face of the opening treatment device 40 by manipulating the surgical knife manipulating portion 43 so as not to damage the channel. While the distal end portion of the opening treatment device 40 is protruding from the endoscope distal end portion 4, the distal end portion is made to protrude from the distal end face of the opening treatment device 40 by manipulating the surgical knife manipulating portion 43. Then, the distal end portion is abutted against the lumen wall 101 having a recess formed thereon. In this state, a high-frequency current is supplied by a high-frequency power source (not shown), and the lumen wall 101 is perforated.

After the lumen wall 101 has been perforated, the opening treatment device 40 is made to further protrude from the endoscope distal end portion 4 as in the above-described embodiment. Then, the sheath 42 of the opening treatment device is inserted into the outside of the lumen wall, i.e., the inside of thoracic cavity or abdominal cavity and the like. After the center portion 63 of the balloon dilator 60 arranged at this sheath 42 is inserted until the center portion has been abutted against the lumen wall 101, the forceps manipulating portion 73 is manipulated to separate the lumen wall 101. Then, a fluid is supplied to the balloon dilator 60 by means of an inflation device (not shown), and the balloon dilator is dilated, whereby an opening in the lumen wall 101 is dilated up to a sufficient size such that the endoscope 1 is inserted.

After sufficient dilation is obtained, the endoscope 1 is inserted into the outside of the lumen wall, i.e., an abdominal cavity or thoracic cavity and the like. Then, the opening treatment device 40 is withdrawn from the forceps opening 5, thereby observing or treating the outside of the lumen wall, i.e., a required site such as abdominal cavity or thoracic cavity.

In a system according to the present embodiment, the channel member 23 is connected to the transparent cap 20, thereby reducing the outer diameter. The other elements are identical to those according to the above-described embodiment.

### Sixth Embodiment

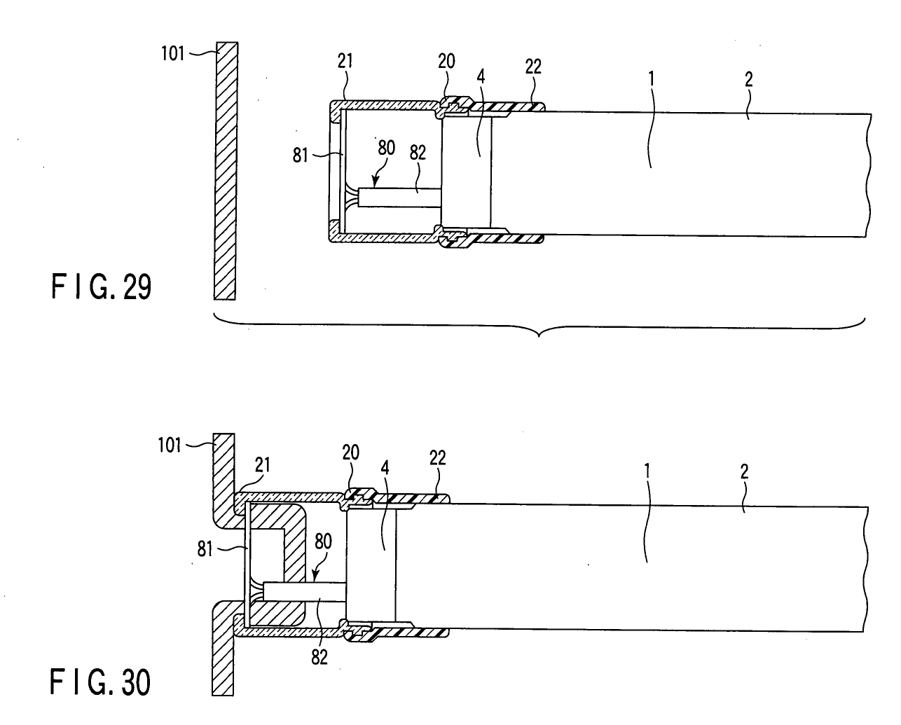

FIG. 29 to FIG. 32 each show a sixth embodiment of the present invention.

A system according to the present embodiment is composed of an endoscope 1; a transparent cap 20 mounted on an endoscope distal end portion 4; and a high-frequency snare 80 inserted into the endoscope 1.

The high-frequency snare 80 is composed of a spare wire 81 and a snare sheath 82; and a manipulating portion and high-frequency power source (not shown).

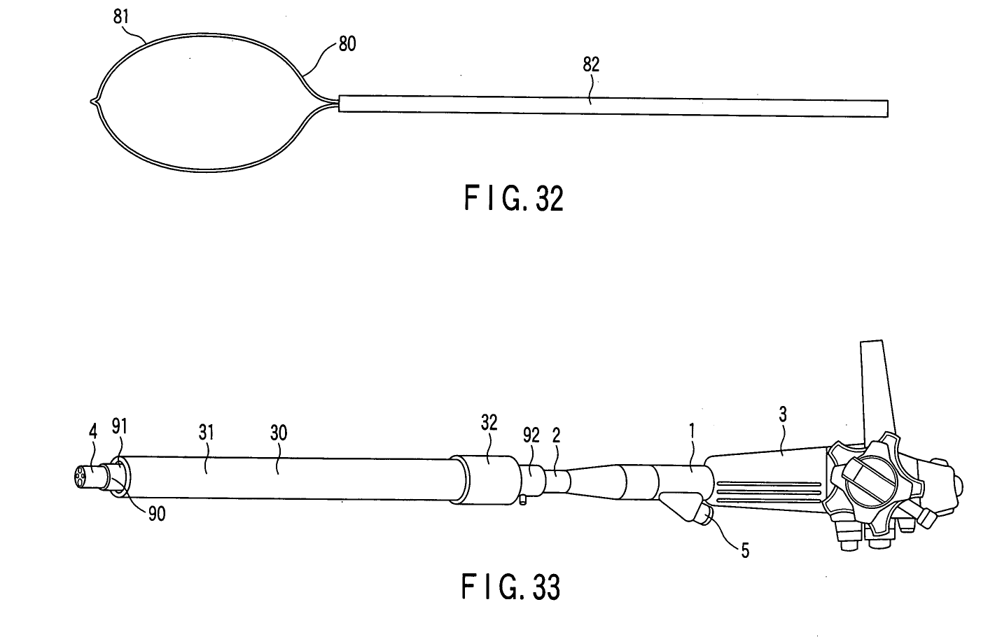

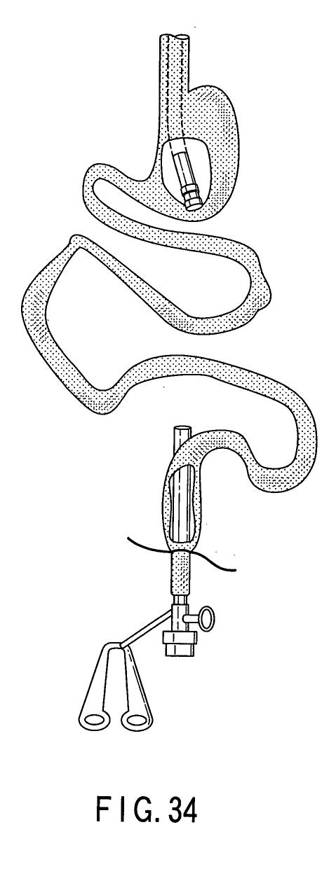

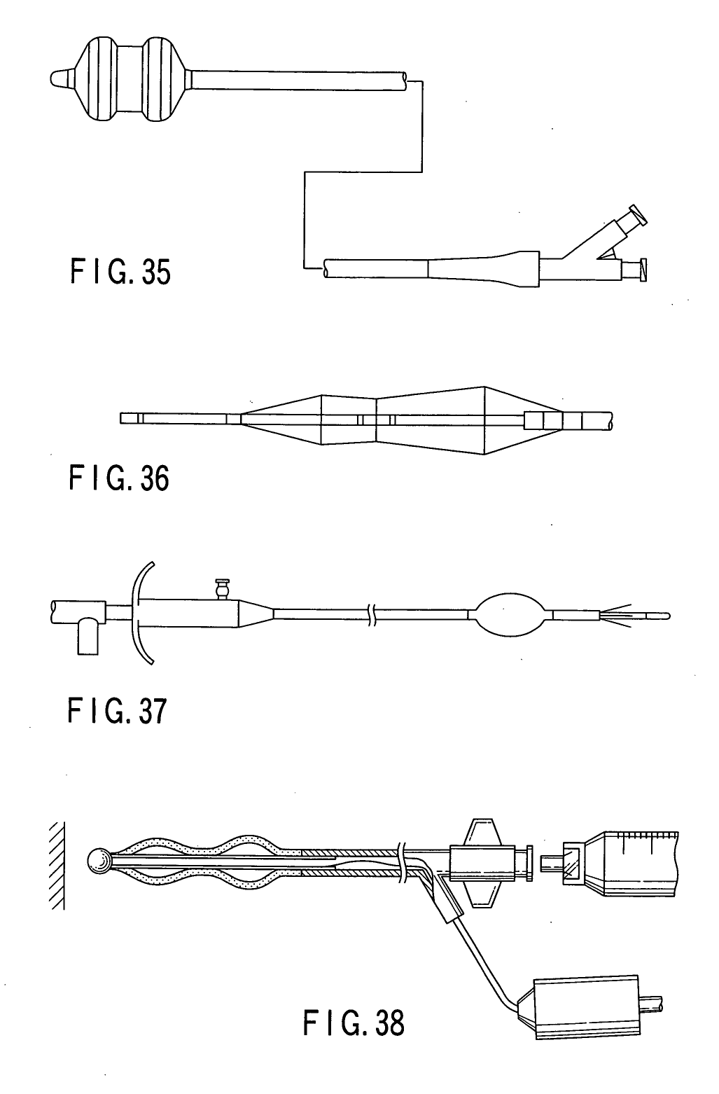

As shown in FIG. 32, the snare wire 81 is formed of a twisted wire made of a metal such as stainless steel, for example, having a loop formed at a distal end portion. The length is 300 mm to 5000 mm to an extent such that the wire can be inserted into the endoscope 1. In particular, it is preferable that the length be 1000 to 2000 mm. The inner diameter of a loop formed at its distal end is substantially 10 mm to 100 mm such that the lumen wall 101 can be tightened. In particular, it is preferable that the inner diameter be 10 mm to 40 mm.

The snare sheath 82 has a tube structure whose cross section is circular, for example. For example, this snare sheath is made of a polymeric resin material similar to the sheath 42, and has at least one bore for passing the snare wire 81. The length is 300 mm to 5000 mm to an extent such that the sheath can be inserted into a body together with the endoscope 1. In particular, it is preferable that the length be 1000 mm to 2000 mm. The inner diameter is 0.4 mm to 20 mm to an extent such that the snare wire 81 passes. In particular, it is preferable that the inner diameter is 0.5 mm to 3 mm.

When this system is used, the endoscope 1 having the transparent cap 20 mounted thereon is first inserted through natural openings of a human body, that is, nose, mouth, anus, vagina, etc. into lumens in the body, that is, esophagus, stomach, duodenum, small intestine, large intestine, rectum, vagina, uterus, etc..

Next, as shown in FIG. 29, the distal end portion 4 fixing the transparent cap 20 is opposed to a required site of the lumen wall 101. Then, the high-frequency snare 80 is inserted into the endoscope 1, and is made to protrude from the distal end portion 4 of the endoscope 1. Then, a loop of the snare wire 81 is formed inside of the transparent cap 20.

In this state, the transparent cap 20 is abutted against the lumen wall 101, and a negative pressure is formed in the transparent cap 20 by means of a suction unit (not shown). In this manner, as shown in FIG. 30, a part of the lumen wall 101 is suctioned into the transparent cap 20 through the loop formed at the snare wire 81, and a recess is formed. Then, the high-frequency snare 80 is tied.

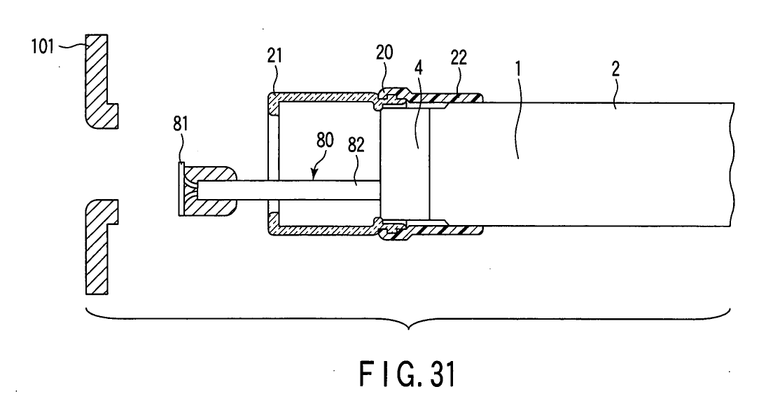

After the snare has been tied, a recess portion is excised with a high-frequency due to the high-frequency energy supplied from the high-frequency power source (not shown) to the snare wire 81. This state is shown in FIG. 31.

After excising the recess portion of the lumen wall 101, the distal end portion 4 of the endoscope 1 is inserted into the outside of the lumen wall, i.e., into the inside of abdominal cavity or thoracic cavity and the like, thereby observing or treating the outside of the lumen wall, i.e., the inside of abdominal cavity or thoracic cavity.

According to the system of the present embodiment, there is no need for a balloon dilator, and thus, simplified surgical operation can be expected.

### Seventh Embodiment

FIG. 33 shows a system according to a seventh embodiment of the present invention.

A system according to the present embodiment comprises an endoscope 1, an over-tube 30, and an introducer 90. This introducer 90 comprises a tubular sheath 91 and a proximal portion 92 connected to the proximal end of this sheath 91.

The introducer 91 has a tube structure whose cross section is circular, and is formed of a polymeric resin material similar to the sheath 42, for example. This sheath 91 has at least one lumen for inserting the endoscope 1. The length is 300 mm to 5000 mm to an extent such that the sheath can reach a target site in a body when it is inserted through a natural opening of a human body. In particular, it is preferable that the length be 1000 mm to 2000 mm. The outer diameter is 3 to 30 mm to an extent such that the sheath can be inserted through a natural opening of a human body. In particular, it is preferable that the outer diameter be 3 to 25 mm. The inner diameter is 3 mm to 30 mm to an extent that the endoscope 1 can be inserted. In particular, it is preferable that the inner diameter be 3 mm to 25 mm.

An introducer proximal portion 92 is formed of a hard pipe shaped member, and is fixed to the introducer sheath 91 by suitable means such as press-fit, ultrasonic wave fusion, thermal fusion, solvent adhesive, or screws and the like, for example. Further, a valve (not shown) for holding the inside of the body from the outside of the body in air tight state is disposed at the introducer proximal portion 92. In this manner, a suction operation or air supply operation can be efficiently made via the endoscope 1, for example.

According to a system of the seventh embodiment, the endoscope 1 inserted into the introducer 90 with the endoscope being further inserted into the over-tube 30 is inserted through natural openings of a human body, that is, nose, mouth, anus, vagina, etc. into lumens in the body, that is, esophagus, stomach, duodenum, small intestine, large intestine, rectum, vagina, uterus, etc.. By using this introducer 90, the accessibility to the inside of abdominal cavity or the inside of thoracic cavity can be improved.

A description of the present invention has been given with reference to preferred embodiments shown in various drawings. Another similar embodiment can be used without deviating from the present invention. In order to attain a function which is identical to that of the present invention, the above-described embodiments can be modified. Therefore, the present invention is not limited to any single embodiment, and various combinations can occur within the intended scope of the present invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1-11. (canceled)

12. A balloon dilator comprising:
a distal end portion;
a center portion;
a proximal end portion;
a distal maximum external diameter portion disposed at a distal side more than the center portion, the distal maximum external diameter portion having a diameter which is greater than the center portion;
a proximal maximum external diameter portion disposed at a proximal side more than the center portion, the proximal maximum external diameter portion having a diameter which is greater than the center portion;
a surface portion; and
a marker provided at the surface portion.

13. A balloon dilator according to claim 12, wherein the marker is an X-ray marker.

14. A balloon dilator according to claim 12, wherein the balloon dilator has a lumen through which a guide wire is inserted.

15. A balloon dilator according to claim 12, wherein the balloon dilator has a high-frequency surgical knife disposed at the distal end portion.

16. A balloon dilator according to claim 15, wherein the high-frequency surgical knife is formed in a needle shape.

17. A balloon dilator according to claim 15, wherein the high-frequency surgical knife can be removed, and a guide wire can be inserted therethrough.

18. A balloon dilator according to claim 16, wherein the high-frequency surgical knife can be removed, a guide wire can be inserted therethrough.

19. A balloon dilator according to claim 12, wherein the marker indicates at least one of the distal maximum diameter portion, center portion, and proximal maximum diameter portion.

20. A balloon dilator comprising:
a sheath having a distal end portion and a proximal end portion;
a dilatable balloon provided on the distal end portion of the sheath, wherein the balloon has a first dilating portion, a second dilating portion, and a third dilating portion provided between the first dilating portion and the second dilating portion, the first and second dilating portions being more dilated than the third dilating portion; and
a marker provided at the balloon or the sheath to identify a position of at least one of the first, second or third dilating portion.

21. The balloon dilator according to claim 20, wherein the marker is configured to identify a position of at least one of the first, second or third expanding portion in a state wherein the balloon is not dilated.

22. The balloon dilator according to claim 20, wherein the marker is provided at the surface of the balloon.

23. The balloon dilator according to claim 20, wherein the balloon is formed by a transparent or translucent material, and the marker is provided at the surface of the sheath.

24. The balloon dilator according to claim 20, wherein a lumen communicating from the distal end portion to the proximal portion is formed on the sheath.

25. The balloon dilator according to claim 20, wherein the marker is an X-ray marker.

26. The balloon dilator according to claim 20, wherein a high-frequency surgical knife is provided on the distal end portion of the sheath.

27. The balloon dilator according to claim 26, wherein the high-frequency surgical knife is formed in a needle shape.

28. The balloon dilator according to claim 26, wherein the high-frequency surgical knife is configured to be removed, and the sheath is configured to insert a guide wire therethrough.

29. The balloon dilator according to claim 24, wherein the lumen is configured to insert a high-frequency surgical knife or a guide wire therethrough.
